(19) United States
(12) Patent Application Publication
(10) Pub. No.: US 2022/0001063 A1
(43) Pub. Date: Jan. 6, 2022

(54) Type of Continuous Disinfection Lighting Lamp

(71) Applicant: Shanghai Jingyun Intelligent Technology Co., Ltd, Shanghai (CN)

(72) Inventors: Yanfei Shen, Shanghai (CN); Xiangning Xiao, Shanghai (CN)

(21) Appl. No.: 16/937,531

(22) Filed: Jul. 23, 2020

(30) Foreign Application Priority Data
Jul. 3, 2020 (CN) CN 202021270384.6

(51) Int. Cl. A61L 2/26; F21V 23/00; F21V 33/00; A61L 2/10; A61L 2/08

(52) U.S. Cl. CPC A61L 2/26; F21V 23/005; F21Y 2115/10; A61L 2/10; A61L 2/084; F21V 33/0068

(57) ABSTRACT

This invention discloses a type of continuous disinfection lighting lamp, which consists of an enclosure, a driving power supply and a lamp board. The above-mentioned lamp board is clamped to the bottom of the enclosure, the above-mentioned driving power supply is located at the upper part or bottom of the lamp board, the above-mentioned bottom of the lamp board is provided with a light source, and the above-mentioned driving power supply and the light source constitute a control connection, and the above-mentioned light source emits the light with the wavelength range between 207 nm to 222 nm, which provides the function of sterilization and disinfection. This lighting lamp provides a continuous disinfection function in addition to the conventional lighting function, and is harmless to human and livestock.

### CROSS REFERENCE TO RELATED APPLICATION

This application claims the priority benefit of Chinese Patent Application No. 202021270384.6, filed on Jul. 3, 2020, which are hereby incorporated by reference in its entirety.

### TECHNICAL FIELD

The utility model refers to the field of lighting lamps, in particular to a kind of continuous disinfection lighting lamp.

### BACKGROUND

White light, a kind of natural light, is essentially an electromagnetic wave. Newton's prism experiment shows that white light can be decomposed into color light band. The color is red, orange, yellow, green, cyan, blue and purple. The range of spectrum is actually the range of electromagnetic waves. The spectrum range of the whole electromagnetic wave is huge, but the range of visible light is very small. It is found that the visible light region is in the wavelength range of 390-770 nm, and other spectra are invisible to human eyes. Electromagnetic wave, which is derived from the perpendicular electric and magnetic fields with the same phase in space, is an electromagnetic field propagating in the form of wave, with wave particle duality. The direction of electric field, magnetic field and propagation of electromagnetic wave are perpendicular to each other, so electromagnetic wave is transverse wave. When the energy level transitions beyond the critical point of radiation, the electromagnetic wave radiates outward in the form of light. At this stage, the wave body is photon. When the frequency of each band is arranged from low to high, they are radio wave, microwave, infrared ray, visible light, ultraviolet ray, X-ray and y ray. The three primary colors of light are red, green and blue. The combination of these three lights can produce the light of other colors, including white light. The effective wavelength range of ultraviolet disinfection can be divided into four different bands: UVA (400˜315 nm), UVB (315˜280 nm), UVC (280˜200 nm) and vacuum ultraviolet (200˜100 nm). Among them, only the parts of UVA and UVB can reach the earth's surface through ozone protection layer and cloud layer. In terms of sterilization rate, UVC is in the range of microbial absorption peak, which can kill viruses and bacteria by destroying the DNA structure of microorganisms within 1 s, while UVA and UVB are very slow because they are outside the range of microbial absorption peak, so it often takes several hours to kill bacteria. Ultraviolet radiation mainly damages microorganisms (bacteria, viruses, fungi and other pathogens) and destroys the function of nucleic acid to kill microorganisms, so as to achieve the purpose of disinfection. The effect of ultraviolet radiation on nucleic acid can lead to the breaking of bonds and chains, crosslinking between strands and the formation of photochemical products, which changes the biological activity of DNA and makes microorganisms unable to replicate themselves. This ultraviolet damage is also lethal damage. However, ultraviolet rays are also harmful to human body. Therefore, it is not suitable for human beings or animals to be present where there is ultraviolet radiation.

The electromagnetic wave with the wavelength of 207 nm to 222 nm constitutes a part of UVC, which is far ultraviolet (Far UVC) and stronger than traditional UVC for disinfection. Its disinfection is physical disinfection, without secondary pollution, and harmless to the environment, ecology and human beings. It can perform disinfection as fast as traditional UVC, and it will not harm human beings. This is the main reason why we invented lamps with continuous disinfection function.

### SUMMARY

In view of above-mentioned problems, the utility model provides a kind of continuous disinfection lighting lamp. This lighting lamp provides a continuous disinfection function in addition to the conventional lighting function, and is harmless to human and livestock.

In order to achieve the above technical purpose, the utility model adopts a kind of continuous disinfection lighting lamp, which consists of an enclosure, a driving power supply and a lamp board. The above-mentioned lamp board is clamped to the bottom of the enclosure, and the above-mentioned driving power supply is located at the upper part or bottom of the lamp board, the bottom of the above-mentioned lamp board is provided with a light source, and the above-mentioned driving power supply and the light source constitute a control connection, and the above-mentioned light source emits far ultraviolet (Far UVC) with the wavelength range between 207 nm to 222 nm, and the light in this wavelength range provides the function of fast sterilization and disinfection.

As a preferred item of the utility model, the wavelength is 210 nm of far ultraviolet (Far UVC).

As a further preferred item of the utility model, the wavelength is 220 nm of far ultraviolet (Far UVC).

As a much further preferred item of the utility model, a percentage of spectral energy for electromagnetic waves in the wavelength range of 207 nm to 222 nm ranges from 5% to 60%.

As a much further preferred item of the utility model, a percentage of spectral energy for electromagnetic waves in the wavelength range of 207 nm to 222 nm accounts for 50%.

As a much further preferred item of the utility model, the above-mentioned light source is a light-emitting diode or gas discharge lamp, or a combination of light-emitting diode and gas discharge lamp.

As a much further preferred item of the utility model, the above-mentioned light source emits white light, with CRI of 90 (min.).

Additionally, the above-mentioned driving power supply and the light source can be an integrated structure with the driving power supply and the light source in one package.

Further additionally, the integrated structure of the above-mentioned driving power supply and the light source includes the bulb or lamp tube.

Further additionally, the integrated structure of the above-mentioned driving power supply and the light source includes the light engine.

Further additionally, the integrated structure of the above-mentioned driving power supply and the light source includes the module.

This invention of lighting lamp emits white light with a wavelength range of 207 nm to 222 nm for far ultraviolet (Far UVC), and its disinfection principle is physical disinfection, which will not produce secondary pollution and is harmless to the environment, ecology and human beings, and is an ideal household lighting device.

### BRIEF DESCRIPTION OF THE DRAWINGS

FIG. 1 shows the structural schematic diagram of the utility model where two kinds of LED light sources are installed on a lamp board;

FIG. 2 shows the structural schematic diagram of the utility model where only one kind of LED light source is installed on a lamp board;

FIG. 3 shows the structural schematic diagram of the utility model where the integrated installation of the driver and the light source and non integrated installation are implemented;

FIG. 4 shows the structural schematic diagram of the utility model where two integrated installations of the driver and the light source are implemented; and

FIG. 5 shows the lamp appearance structure diagram of the utility model.

### DETAILED DESCRIPTION

The utility model is further described in combination with the attached figures and the specific implementation.

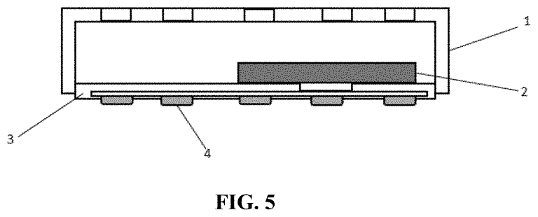

It can be seen from FIG. 5, a kind of continuous disinfection lighting lamp, which consists of an enclosure1, a driver2 and a lamp board3. From the above-mentioned structure, lamp board3 is clamped to the bottom of the enclosure1, the driver2 is located at the upper part of the lamp board3, it can also be located at the bottom of the lamp board3, and the bottom of lamp board3 is provided with a light source4, and the driver2 and the light source4 constitute a control connection. For this invention, a special light source4 emits far ultraviolet (Far UVC) with the wavelength range between 207 nm to 222 nm, and the light in this wavelength range provides the function of fast sterilization and disinfection.

As a preferred item of the utility model, a special light source4 emits far ultraviolet (Far UVC) with the wavelength of 210 nm, within this range, the sterilization effect of wavelength is better.

As another preferred item of the utility model, a special light source4 emits far ultraviolet (Far UVC) with the wavelength of 220 nm, within this range, the sterilization effect of wavelength is better.

As a preferred item of the utility model, a percentage of spectral energy for electromagnetic waves in the wavelength range of 207 nm to 222 nm ranges from 5% to 60%, and in practical application, we found that the electromagnetic wave in the far ultraviolet (Far UVC) range of 207 nm to 222 nm accounts for 50% of spectral energy.

As a preferred item of the utility model, the mentioned light source4 is a light-emitting diode or gas discharge lamp, or a combination of light-emitting diode and gas discharge lamp, or other light sources with lighting effects, all of which are within the protection scope of the utility model.

As a preferred item of the utility model, the mentioned light source4 emits white light, with Color Rendering Index (CRI) of 90 (min.) in practical application.

As a preferred item of the utility model, the mentioned driver2 and light source4 can be an integrated structure with the driver2 and light source4 in one package, and in practical application, the integrated structure of mentioned driver2 and the light source4 includes the bulb or lamp tube, or includes the light engine, or includes the module.

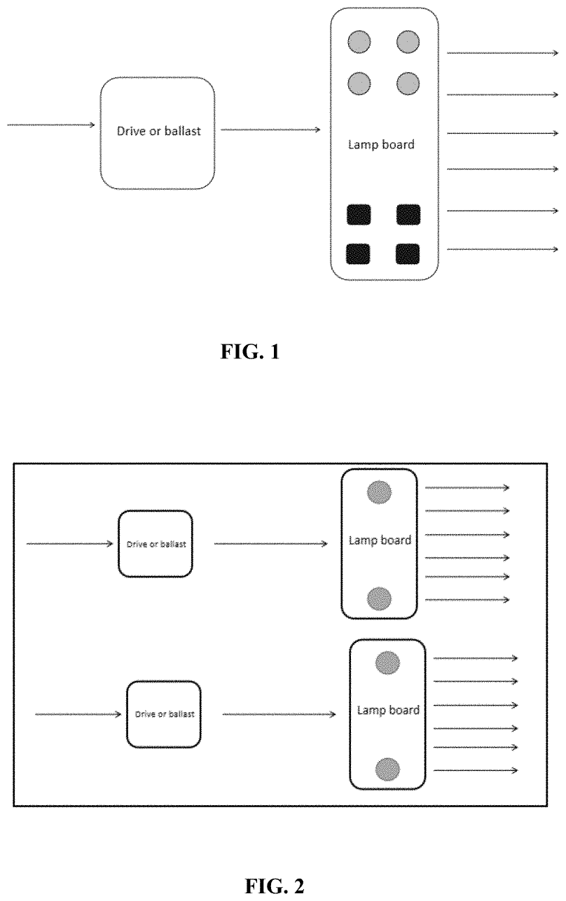

In practical application, two kinds of LED light sources can be installed on the lamp board3, one is ordinary LED light source, which does not provide sterilization effect, and the other is LED light source with sterilization effect mentioned in this invention (see FIG. 1).

In practical application, one kind of LED light source can be installed on the lamp board3, i.e. the one with sterilization effect mentioned in this invention (see FIG. 2).

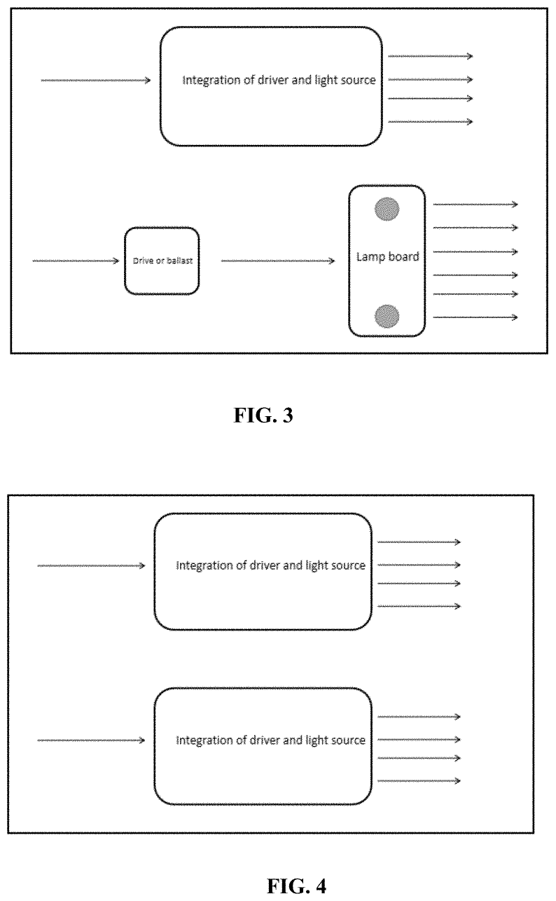

As mentioned above, this utility model can be the integrated structure of the driver and the light source, or the non integrated structure of the driver and the light source, and FIG. 3 shows the structural schematic diagram of the integrated installation of the driver and the light source and the non integrated installation (see FIG. 3); or it can also be two integrated installations of the driver and the light source (see FIG. 4), or two more integrated installations of the driver and the light source, all of which are within the protection scope of the utility model.

Generally, this utility model of lighting lamp emits white light with a wavelength range of 207 nm to 222 nm for far ultraviolet (Far UVC), and its disinfection principle is physical disinfection, which will not produce secondary pollution and is harmless to the environment, ecology and human beings, and is an ideal household lighting device.

The above is only the preferred implementation of the utility model. It should be pointed out that ordinary technical personnel in the technical field can make several improvements and modifications without departing from the technical principle of the utility model, and these improvements and modifications shall also be regarded as the protection scope of the utility model.

What is claimed is:

1. A continuous disinfection lighting lamp, comprising:
an enclosure;
a driving power supply;
a lamp board; and
a light source;
wherein the lamp board is clamped to a bottom of the enclosure;
wherein the driving power supply is located at an upper part or the bottom of the lamp board;
wherein the bottom of the lamp board is provided with the light source,
wherein the driving power supply and the light source constitute a control connection, configured to control the light source to emit far ultraviolet (Far UVC) with a wavelength range between 207 nm to 222 nm, and the light in this wavelength range provides the function of sterilization and disinfection.

2. The continuous disinfection lighting lamp according to the claim 1, wherein the wavelength of the Far UVC is 210 nm.

3. The continuous disinfection lighting lamp according to the claim 1, wherein the wavelength of the Far UVC is 220 nm.

4. The continuous disinfection lighting lamp according to the claim 1, wherein a percentage of spectral energy for electromagnetic waves in the wavelength range of 207 nm to 222 nm ranges from 5% to 60%.

5. The continuous disinfection lighting lamp according to the claim 1, wherein the light source is chosen from the group of a light-emitting diode, a gas discharge lamp, or a combination of light-emitting diode and gas discharge lamp.

6. The continuous disinfection lighting lamp according to the claim 1, wherein the light source emits white light, with CRI of 90 (minutes).

7. The continuous disinfection lighting lamp according to the claim 1, wherein an integrated structure integrates the driving power supply and the light source into one package.

8. The continuous disinfection lighting lamp according to the claim 7, wherein the integrated structure further comprises a bulb or lamp tube.

9. The continuous disinfection lighting lamp according to the claim 8, wherein the integrated structure further comprises a light engine.

10. The continuous disinfection lighting lamp according to the claim 8, wherein the integrated structure comprises a control module.
